(19) United States
(12) Patent Application Publication
(10) Pub. No.: US 2019/0298445 A1
(43) Pub. Date: Oct. 3, 2019

(54) LIQUID COOLING SYSTEM FOR NEUTRAL ELECTRODE

(71) Applicant: BIOSENSE WEBSTER (ISRAEL) LTD., Yokneam (IL)

(72) Inventor: Mikhael Feldchtein, Kiryat Yam (IL)

(21) Appl. No.: 15/936,526

(22) Filed: Mar. 27, 2018

(51) Int. Cl. A61B 18/16; A61B 18/14; A61B 18/12

(52) U.S. Cl. CPC A61B 18/16; A61B 2018/00011; A61B 18/1233; A61B 18/1492

(57) ABSTRACT

Described embodiments include an apparatus that includes at least one electrically-conducting plate, configured for placement on a body of a subject, and one or more tubes coupled to a surface of the electrically-conducting plate, the tubes being configured to carry a fluid over the electrically-conducting plate while the electrically-conducting plate is on the body of the subject and electric current passes through the electrically-conducting plate. Other embodiments are also described.

### FIELD OF THE INVENTION

The present invention relates to medical procedures that involve the passage of electric current through biological tissue.

### BACKGROUND

In some medical procedures, such as unipolar cardiac ablation procedures, electric current is passed between a first electrode, which is in contact with internal tissue of a subject, and a second electrode, which is coupled to the surface of the body of the subject. The second electrode may be referred to as a “neutral electrode,” a “return electrode,” or an “indifferent electrode.”

U.S. Patent Application Publication 2009/0171341 describes apparatus and methods for performing electrosurgery on a patient by evenly distributing electric current density at a return electrode unit having a plurality of concentric return electrodes. In an embodiment, each electrode may be independently coupled to a passive electrical element, and each of the passive electrical elements may have a different value of capacitance, resistance or inductance, according to the configuration of the concentric return electrodes, to provide the even distribution of electric current density between the plurality of concentric return electrodes of the return electrode unit.

### SUMMARY OF THE INVENTION

There is provided, in accordance with some embodiments of the present invention, an apparatus that includes at least one electrically-conducting plate, configured for placement on a body of a subject. The apparatus further includes one or more tubes coupled to a surface of the electrically-conducting plate, the tubes being configured to carry a fluid over the electrically-conducting plate while the electrically-conducting plate is on the body of the subject and electric current passes through the electrically-conducting plate.

In some embodiments, the tubes include a first tube-end and a second tube-end that protrude from the electrically-conducting plate, and the tubes are configured to carry the fluid, over the plate, from the first tube-end to the second tube-end.

In some embodiments, the first tube-end and the second tube-end include respective threaded connectors.

In some embodiments, the tubes are interconnected, such as to define a tube network.

In some embodiments, the tubes include:

a first tube coupled to a first edge of the surface;

a second tube coupled to a second edge of the surface that is opposite the first edge; and

a plurality of third tubes that connect the first tube to the second tube, such that the fluid flows between the first tube and the second tube via the third tubes.

In some embodiments, the apparatus further includes one or more temperature sensors coupled to the electrically-conducting plate.

There is further provided, in accordance with some embodiments of the present invention, an apparatus for use with a patch that includes an electrically-conducting plate. The apparatus includes one or more tubes, configured to carry a fluid over the electrically-conducting plate while the patch is coupled to a body of a subject, and while electric current passes through the electrically-conducting plate. The apparatus further includes an adhesive sheet, including an inner adhesive portion, configured to couple to the tubes, and an outer adhesive portion, which at least partly surrounds the inner adhesive portion and is configured to couple the tubes to a surface of the electrically-conducting plate by adhering to the patch while the tubes are coupled to the inner adhesive portion. The apparatus further includes an inner adhesive-sheet backing, which covers the inner adhesive portion, and an outer adhesive-sheet backing, which covers the outer adhesive portion.

In some embodiments, the apparatus further includes a tube-mounting sheet, the tubes are mounted on the tube-mounting sheet, and the inner adhesive portion is configured to couple to the tubes by adhering to the tube-mounting sheet.

In some embodiments, the apparatus further includes one or more temperature sensors coupled to the tube-mounting sheet.

In some embodiments,

the outer adhesive portion is a first outer adhesive portion and the outer adhesive-sheet backing is a first outer adhesive-sheet backing,

the adhesive sheet further includes a second outer adhesive portion, which at least partly surrounds the first outer adhesive portion and is configured to adhere to skin of the subject, and

the apparatus further includes a second outer adhesive-sheet backing, which covers the second outer adhesive portion.

In some embodiments, the tubes are interconnected, such as to define a tube network.

In some embodiments, the tubes include:

a first tube;

a second tube that is opposite the first tube; and

a plurality of third tubes that connect the first tube to the second tube.

There is further provided, in accordance with some embodiments of the present invention, a method that includes connecting a first fluid conduit to a first tube-end of one or more tubes coupled to a surface of an electrically-conducting plate, connecting a second fluid conduit to a second tube-end of the tubes, and, while the electrically-conducting plate is on a body of a subject and electric current passes through the electrically-conducting plate, passing a fluid from the first fluid conduit, through the tubes, to the second fluid conduit, such that the fluid evacuates heat from the electrically-conducting plate.

In some embodiments, passing the fluid through the tubes includes passing the fluid through the tubes by, using a pump, pumping the fluid from a fluid source through the first fluid conduit, such that the fluid flows through the first fluid conduit, through the tubes, and through the second fluid conduit to a drain.

In some embodiments, passing the fluid through the tubes includes passing the fluid through the tubes by cyclically pumping the fluid through the tubes.

In some embodiments, cyclically pumping the fluid through the tubes includes cyclically pumping the fluid through the tubes by cyclically pumping the fluid from a fluid bag through the first fluid conduit, such that the fluid flows through the first fluid conduit, through the tubes, and through the second fluid conduit to the fluid bag.

In some embodiments, the method further includes cooling the fluid while the fluid flows through the second fluid conduit.

In some embodiments,

the tubes include:
a first tube that runs along a first edge of the surface and terminates at the first tube-end,
a second tube that runs along a second edge of the surface, which is opposite the first edge, and terminates at the second tube-end, and
a plurality of third tubes that connect the first tube to the second tube, and

passing the fluid through the tubes including passing the fluid between the first tube and the second tube via the third tubes.

In some embodiments, the plate is included in a patch, and the method further includes, prior to passing the fluid through the tubes, adhering the patch to skin of the subject.

In some embodiments, the method further includes coupling the tubes to the surface of the electrically-conducting plate, by adhering an adhesive sheet, over the tubes, to the patch.

In some embodiments, the method further includes, prior to coupling the tubes to the surface of the electrically-conducting plate, coupling the adhesive sheet to the tubes.

In some embodiments, the tubes are mounted on a tube-mounting sheet, and coupling the adhesive sheet to the tubes includes coupling the adhesive sheet to the tubes by adhering the adhesive sheet to the tube-mounting sheet.

In some embodiments, the method further includes adhering the adhesive sheet to skin of the subject.

In some embodiments, passing the fluid through the tubes includes controlling a rate at which the fluid is passed through the tubes responsively to a sensed temperature of the electrically-conducting plate.

The present invention will be more fully understood from the following detailed description of embodiments thereof, taken together with the drawings, in which:

### BRIEF DESCRIPTION OF THE DRAWINGS

FIG. 1 is a schematic illustration of a system for ablating tissue of a subject, in accordance with some embodiments of the present invention;

FIGS. 2A-C are schematic illustrations of different respective cooling systems for a neutral electrode patch, in accordance with some embodiments of the present invention;

FIGS. 3A-B are schematic illustrations of a neutral electrode patch having an integrated cooling system, in accordance with some embodiments of the present invention;

FIG. 4 is a schematic illustration of cooling apparatus for use with a neutral electrode patch, in accordance with some embodiments of the present invention; and

FIG. 5 is a flow diagram for a method for beginning an ablation procedure, in accordance with some embodiments of the present invention.

### DETAILED DESCRIPTION OF EMBODIMENTS

### Overview

During some procedures, a large amount of electric current may pass through a neutral electrode, causing a large increase in the temperature of the neutral electrode. This increase in temperature may place the subject at risk of a burn. Hypothetically, this challenge might be addressed by increasing the size of the neutral electrode, and/or using multiple neutral electrodes. However, these solutions are not practical in most cases.

Embodiments of the present invention therefore address the aforementioned challenge, by providing a liquid cooling system for the neutral electrode. The system comprises one or more tubes that carry a fluid, such as water, over the surface of the electrode during the procedure. As the fluid flows over the electrode, the fluid evacuates heat from the electrode. The system may implement a closed loop, in that the same fluid is continually cycled through the system, or an open loop.

In some embodiments, the tubes are integrated with the neutral electrode in a single, integrated neutral electrode patch. For example, the integrated patch may comprise a network of interconnected tubes coupled to an electrically-conducting plate that serves as the neural electrode, with two tube-ends, which serve as the inlet and outlet ports of the network, protruding from the plate. The tubes may be covered, e.g., by a permanent plastic cover, leaving only the two tube-ends exposed. Prior to coupling the patch to the subject, the plate may be connected to a generator, and the tube-ends may be connected to fluid conduits. Subsequently, the patch may be coupled to the subject, e.g., by sticking an adhesive patch-sheet, which is coupled to the plate, onto the subject.

In other embodiments, the tubes belong to a separate, reusable cooling unit comprising, for example, a network of interconnected tubes affixed to a flexible piece of material (e.g., nylon). A sticker, configured to stick the cooling unit onto the electrode patch, is further provided. Prior to the procedure, an inner portion of the sticker is stuck onto the top surface of the cooling unit. Next, an outer portion of the sticker is stuck onto the top surface of the adhesive patch-sheet that is coupled to the electrode plate, thus pressing the tubes against the plate, or against the portion of the adhesive patch-sheet that is stuck to the plate. Subsequently, after connecting the network of tubes to the fluid conduits, and connecting the plate to the generator, the patch is attached to the subject, as described above.

### System Description

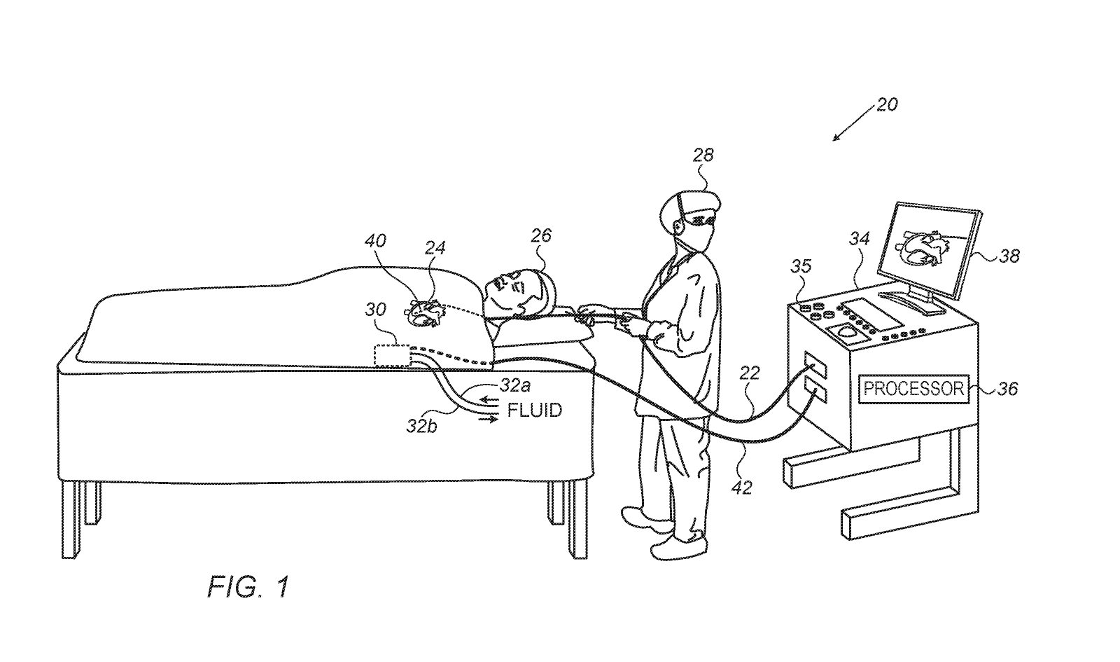

Reference is initially made to FIG. 1, which is a schematic illustration of a system 20 for ablating tissue of a subject 26, in accordance with some embodiments of the present invention.

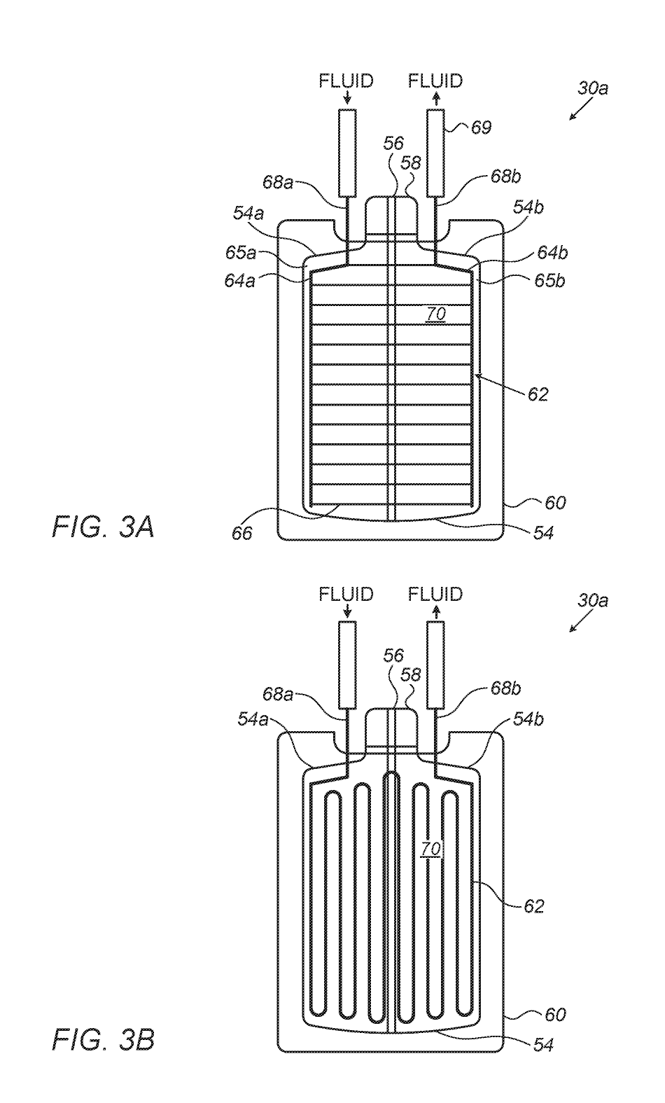

FIG. 1 depicts a physician 28 performing a unipolar ablation procedure on subject 26, using an ablation catheter 22. In this procedure, physician 28 first inserts the distal tip 40 of catheter 22 into the subject, and then navigates distal tip to the tissue that is to be ablated. For example, the physician may advance the distal tip through the vasculature of the subject until the distal tip is in contact with tissue located within the heart 24 of the subject. Next, while distal tip 40 contacts the tissue, the physician causes radiofrequency (RF) electric currents to be passed between one or more electrodes on distal tip 40 and a neutral electrode patch 30 that is coupled externally to the subject, e.g., to the subject's back. While the RF currents pass through neutral electrode patch 30, a fluid, such as water, is delivered to the neutral electrode patch via a first fluid conduit 32a. As further described below with reference to the subsequent figures, this fluid evacuates heat from the neutral electrode patch, carrying the heat away via a second fluid conduit 32b. First fluid conduit 32a and second fluid conduit 32b typically comprise respective tubes, which may be coupled to patch 30 as further described below with reference to FIGS. 3A-B.

Typically, catheter 22 is connected to a console 34 comprising controls 35, which are used by the physician to control the parameters of the ablating currents. In particular, in response to the manipulation of controls 35 by physician 28, a processor 36 adjusts the parameters of the ablating currents, by outputting appropriate instructions to the signal generator that generates the currents. (In some embodiments, processor 36 is an internal processor belonging to the signal generator.) Electrode patch 30 may also be connected to console 34, via at least one wire 42. In some embodiments, system 20 further comprises a display 38, and processor 36 causes display 38 to display relevant output to physician 28 during the procedure.

Notwithstanding the particular type of procedure depicted in FIG. 1, it is noted that the embodiments described herein may be applied to any suitable type of procedure that requires a neutral electrode, such as, for example, an electrosurgical procedure.

In general, processor 36 may be embodied as a single processor, or as a cooperatively networked or clustered set of processors. Processor 36 is typically a programmed digital computing device comprising a central processing unit (CPU), random access memory (RAM), non-volatile secondary storage, such as a hard drive or CD ROM drive, network interfaces, and/or peripheral devices. Program code, including software programs, and/or data are loaded into the RAM for execution and processing by the CPU and results are generated for display, output, transmittal, or storage, as is known in the art. The program code and/or data may be downloaded to the computer in electronic form, over a network, for example, or it may, alternatively or additionally, be provided and/or stored on non-transitory tangible media, such as magnetic, optical, or electronic memory. Such program code and/or data, when provided to the processor, produce a machine or special-purpose computer, configured to perform the tasks described herein.

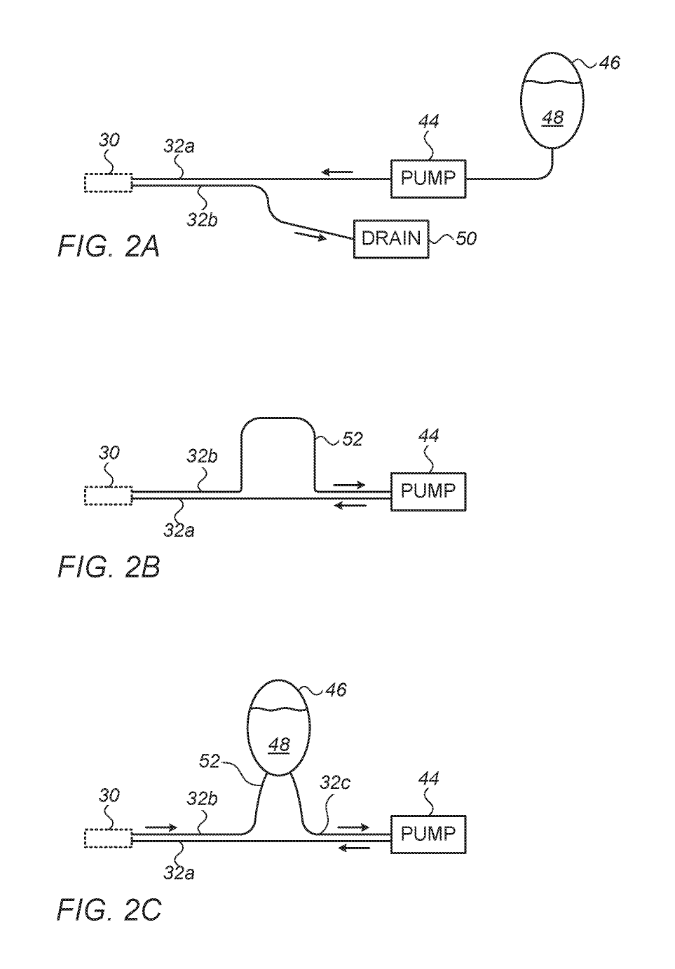

Reference is now made to FIGS. 2A-C, which are schematic illustrations of different respective cooling systems for neutral electrode patch 30, in accordance with some embodiments of the present invention.

FIG. 2A shows a pump 44 pumping a cooling fluid 48 from a fluid source, such as a fluid bag 46 or a tap, through first fluid conduit 32a to electrode patch 30. (In some embodiments, pump 44 is contained within console 34 (FIG. 1).) The rate at which the fluid is pumped may be constant, or it may, alternatively, be continually adjusted by processor 36 responsively to the temperature of the electrode, as further described below with reference to FIGS. 3A-B. Processor 36 may be connected to pump 44 over any suitable wired or wireless communication interface, via which the processor may control the pump.

As further described below with reference to FIGS. 3A-B, fluid 48 flows through tubes in electrode patch 30, thus collecting heat from the electrode. From electrode patch 30, fluid 48 flows through second fluid conduit 32b to a drain 50. FIG. 2A thus illustrates an open-loop cooling system, in that any fluid that leaves the electrode patch is not reused for further cooling of the patch.

FIGS. 2B-C, on the other hand, illustrate a closed-loop cooling system, in that a predetermined amount of fluid continually circulates through the system, without the further addition of fluid to the system. In particular, in FIGS. 2B-C, pump 44 cyclically pumps the fluid through the tubes in electrode patch 30. In such embodiments, second fluid conduit 32b typically comprises a cooling loop 52, along which heat is transferred from fluid 48 to a surrounding medium (e.g., to the surrounding air), such that the temperature of the fluid drops below that of the electrode.

In some embodiments, the fluid is actively cooled while the fluid flows through second fluid conduit 32b. For example, one or more cooling fans, Peltier coolers, or other cooling elements may be disposed along cooling loop 52. Alternatively or additionally, as shown in FIG. 2C, fluid bag 46 may be disposed along cooling loop 52. In such embodiments, fluid bag 46 has two ports, one port being connected to second fluid conduit 32b, and the other port being connected to a third conduit 32c that leads to the pump. Thus, fluid 48 is pumped from fluid bag 46 through the first fluid conduit, such that the fluid flows through the first fluid conduit, through the tubes in electrode patch 30, and through the second fluid conduit to the fluid bag. Upon returning to the fluid bag, the heated fluid rises to the top of the bag, while the cooler fluid flows out of the bag and into third conduit 32c. The heated fluid is then cooled by the cooler fluid that remains in fluid bag 46.

Reference is now made to FIGS. 3A-B, which are schematic illustrations of a neutral electrode patch 30a having an integrated cooling system, in accordance with some embodiments of the present invention. Electrode patch 30a is an embodiment of electrode patch 30 (FIGS. 2A-C) in which one or more tubes 62, which are configured to carry fluid 48 through the patch, are integrated into the patch during the manufacturing process.

Electrode patch 30a comprises at least one electrically-conducting plate 54, configured for placement on the body of subject 26. Plate 54 may comprise any suitable conducting metal, such as copper or aluminum, or non-metal, such as an intrinsically conducting polymer. In some embodiments, plate 54 comprises two or more portions that are electrically-insulated from each other. Such a configuration may facilitate verifying that the plate is in electrical contact with the body of the subject, in that a small test voltage may be applied between various pairs of portions of the plate. For example, as shown in FIGS. 3A-B, plate 54 may comprise two portions: a first portion 54a, and a second portion 54b that is separated from first portion 54a by a strip 56 of electrically-insulating material.

Plate 54 functions as a neutral electrode, in that, while plate 54 is on the body of the subject, a voltage is applied between the plate and an electrode on distal tip 40 (FIG. 1), such that electric current passes through the plate. Typically, plate 54 comprises at least one electrical connector 58, such as a pin-and-socket connector, which facilitates connecting plate 54 to the generator, e.g., via wire 42 (FIG. 1). Plate 54 may comprise a respective connector for each portion of the plate, with a different respective wire connecting each portion to the generator.

Typically, patch 30a further comprises an adhesive patch-sheet 60, which covers the top surface 70 of plate 54. (The top surface of the plate is the surface that faces away from the subject when the plate is coupled to the subject.) Adhesive patch-sheet 60 further extends beyond the edges of plate 54, such that the adhesive patch-sheet may couple patch 30a to the subject by adhering to skin of the subject. (Although, to facilitate the description herein, adhesive patch-sheet 60 is drawn transparently, it is noted that, in practice, adhesive patch-sheet 60 is not necessarily transparent.)

Tubes 62 are integrated with plate 54 by virtue of being coupled to surface 70. For example, tubes 62 may be glued onto surface 70, e.g., with adhesive patch-sheet 60, and/or another layer of material (such as a firm plastic cover), covering the tubes. Alternatively, tubes 62 may be pressed against surface 70 by adhesive patch-sheet 60, even without necessarily being glued to the surface. As yet another alternative, tubes 62 may be glued onto adhesive patch-sheet 60, e.g., with another layer of material covering the tubes. Tubes 62 are configured to carry fluid 48 (FIGS. 2A-C) over plate 54 while the plate is on the body of the subject and electric current passes through the plate, such that the fluid evacuates at least some of the heat that is generated by the passage of electric current through the plate.

In general, tubes 62 may be made of any suitable material. Typically, however, for safety, tubes 62 are made of an electrically-insulating material, such as Nylon or any other suitable thermal conductor.

Typically, tubes 62 comprise a first tube-end 68a and a second tube-end 68b that protrude from the plate, and the tubes carry the fluid, over plate 54, from first tube-end 68a to second tube-end 68b. First tube-end 68a and second tube-end 68b comprise respective connectors 69 configured to connect to first fluid conduit 32a and second fluid conduit 32b, respectively. For example, each of the tube-ends may comprise a male or female threaded connector, and each of the fluid conduits may comprise a complementary threaded ending, such that the tube-ends may be screwed into the fluid conduits, or vice versa.

In some embodiments, as shown in FIG. 3A, tubes 62 are interconnected, such as to define a tube network that covers a relatively large portion of surface 70. For example, the tubes may comprise a first main tube 64a coupled to a first edge 65a of surface 70, a second main tube 64b coupled to a second edge 65b of surface 70 that is opposite first edge 65a, and a plurality of auxiliary tubes 66, which typically have a smaller diameter than that of first main tube 64a and second main tube 64b, that connect the first main tube to the second main tube. (The first main tube terminates at first tube-end 68a, while the second main tube terminates at second tube-end 68b.) Fluid thus flows between the first main tube and the second main tube via the auxiliary tubes.

In other embodiments, as shown in FIG. 3B, patch 30a comprises a single tube that is twisted back and forth (or “snaked”) over surface 70, such as to cover a relatively large portion of the surface.

In some embodiments, one or more temperature sensors (not shown) are coupled to plate 54, and processor 36 (FIG. 1) controls the rate at which fluid passes through tubes 62—e.g., by controlling the rate at which pump 44 (FIGS. 2A-C) pumps the fluid—responsively to the temperature of plate 54 that is sensed by the temperature sensors. In particular, in response to a higher sensed temperature, the processor may increase the rate of fluid flow, and vice versa.

For example, a thermocouple matrix may be coupled to surface 70, e.g., as described in U.S. Patent Application Publication 2008/0281310. Such a matrix may comprise a first plurality of wires, made of a first metal (e.g., copper), laid in a first direction across surface 70, and a second plurality of wires, made of a second metal (e.g., constantan), laid in a second direction that is perpendicular to the first direction, such that the voltage produced at the junctions between the first wires and second wires changes with temperature. Processor 36 may receive the junction voltages, and, in response to these voltages (which indicate the temperature of the plate), control the rate of fluid flow.

It is noted that the respective shapes of plate 54 and adhesive patch-sheet 60 depicted in FIGS. 3A-B are provided by way of example only. In practice, each of these elements may have any suitable shape.

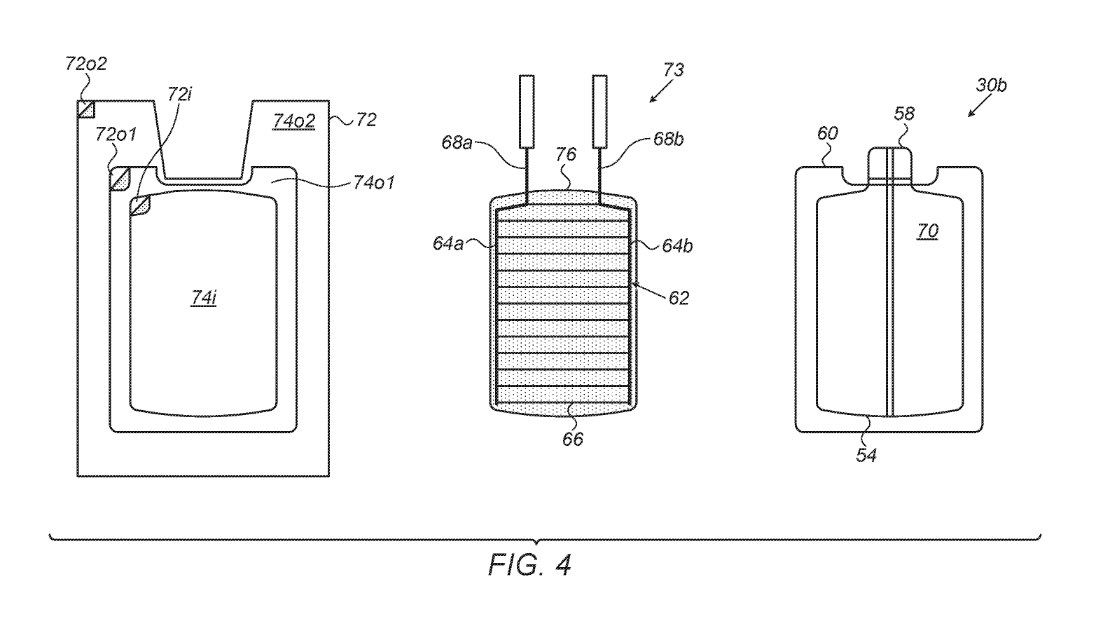

Reference is now made to FIG. 4, which is a schematic illustration of cooling apparatus for use with a neutral electrode patch 30b, in accordance with some embodiments of the present invention.

In general, electrode patch 30b is similar to electrode patch 30a (FIGS. 3A-B), except for the fact that tubes 62 are not integrated into the patch during the manufacturing process. Rather, tubes 62 are included in a separate tube unit 73, which is reversibly couplable to patch 30b. An advantage of this embodiment is that the tubes may be used with multiple neutral electrode patches. Before each procedure, tube unit 73 is coupled to the relevant patch using an adhesive sheet 72, as described in detail below. After the procedure, the tube unit is decoupled from the patch, and the patch and adhesive sheet 72—but not the tube unit—are discarded.

In some embodiments, tube unit 73 comprises a tube-mounting sheet 76, comprising any suitable material (e.g., Nylon), and tubes 62 are mounted onto tube-mounting sheet 76, e.g., by virtue of being glued onto the tube-mounting sheet. In such embodiments, adhesive sheet 72 couples to the tubes by adhering to tube-mounting sheet 76, as further described below. One or more temperature sensors, such as the thermocouple matrix described above, may be coupled (e.g., glued) to tube-mounting sheet 76.

In other embodiments, tube unit 73 does not comprise a tube-mounting sheet, and adhesive sheet 72 couples to the tubes by adhering directly to the tubes.

As in integrated electrode patch 30a, tubes 62 may be interconnected such as to define a tube network, as described above with reference to FIG. 3A. For example, the tubes may comprise first main tube 64a, second main tube 64b, which is opposite the first main tube, and auxiliary tubes 66, which connect the first main tube to the second main tube. Alternatively, tubes 62 may be interconnected in any other suitable arrangement, or a single tube may be repeatedly twisted back and forth, as described above with reference to FIG. 3B. As in the case of FIGS. 3A-B, tubes 62 may be connected to fluid conduits 32a-b via tube-ends 68a-b, respectively. Typically, the size and shape of the area that is covered by tubes 62 (excluding the tube-ends, which are configured to protrude from plate 54 following the coupling of tubes 62 to the plate), are approximately the same as the size and shape of plate 54 (excluding electrical connector 58).

Adhesive sheet 72 comprises multiple adhesive portions, which are covered by respective adhesive-sheet backings. In particular, the adhesive sheet comprises an inner adhesive portion 72i, which is covered by an inner adhesive-sheet backing 74i, and at least one outer adhesive portion, which at least partly surrounds inner adhesive portion 72i. For example, the adhesive sheet may comprise a first outer adhesive portion 72o1, which is covered by a first outer adhesive-sheet backing 74o1, and a second outer adhesive portion 72o2, which at least partly surrounds first outer adhesive portion 72o1 and is covered by a second outer adhesive-sheet backing 74o2.

Inner adhesive portion 72i, which is typically approximately the same size and shape as conducting plate 54 (excluding electrical connector 58), couples to tube unit 73. For example, if the tube unit comprises tube-mounting sheet 76, the inner adhesive portion may adhere to the top surface of tube-mounting sheet 76, i.e., the surface of the tube-mounting sheet that is opposite the surface on which the tubes are mounted and that faces away from the subject. Alternatively, if the tube unit does not comprise tube-mounting sheet 76, the inner adhesive portion may adhere directly to tubes 62.

First outer adhesive portion 72o1 couples the tubes to surface 70 by adhering to patch 30b while the tubes are coupled to inner adhesive portion 72i. For example, first outer adhesive portion 72o1 may be approximately the same size and shape as adhesive patch-sheet 60, and may couple the tubes to surface 70 by adhering to adhesive patch-sheet 60. Second outer adhesive portion 72o2, if included in adhesive sheet 72, adheres to skin of the subject, such as to strengthen the coupling between the tubes and the patch.

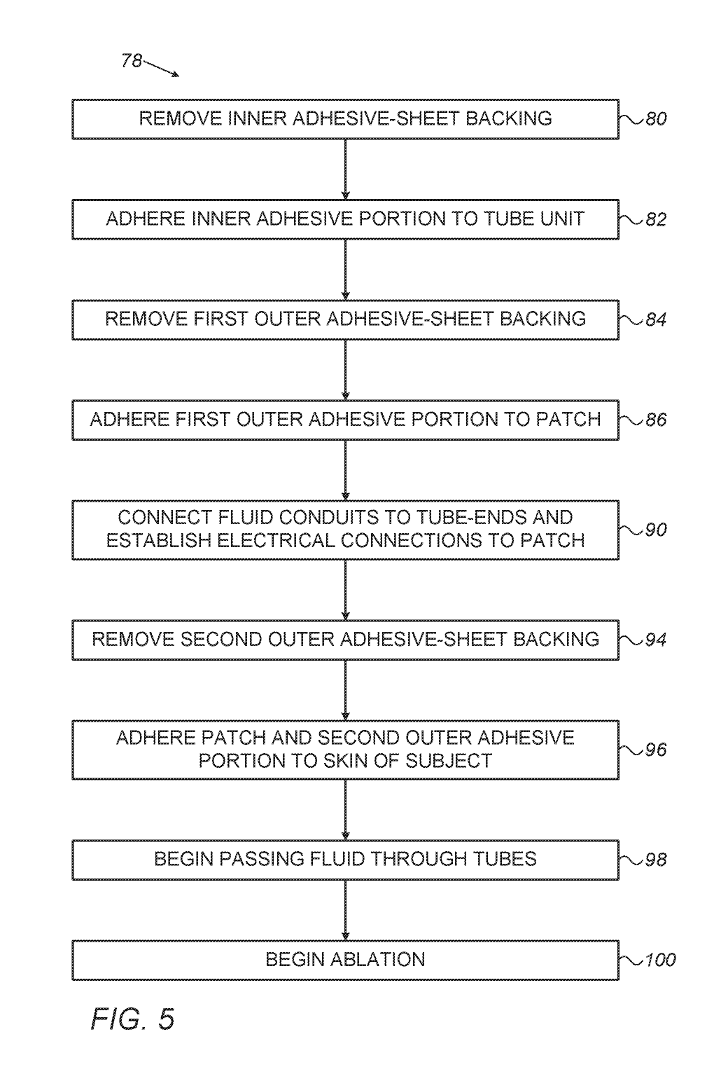

Reference is now made to FIG. 5, which is a flow diagram for a method 78 for beginning an ablation procedure, in accordance with some embodiments of the present invention.

FIG. 5 assumes that the tubes are not integrated with the electrode, but rather, adhesive sheet 72 is used to couple the tubes to the electrode, as described above with reference to FIG. 4. Accordingly, method 78 begins with a first removing step 80, at which inner adhesive-sheet backing 74i is removed from the inner adhesive portion of adhesive sheet 72. Next, at a first adhering step 82, the inner adhesive portion is coupled to tubes 62 by adhering the inner adhesive portion to tube unit 73. For example, the inner adhesive portion may be adhered to tube-mounting sheet 76, or directly to the tubes.

Subsequently, at a second removing step 84, first outer adhesive-sheet backing 74o1 is removed from the first outer adhesive portion of adhesive sheet 72. Next, at a second adhering step 86, the first outer adhesive portion is adhered, over tube unit 73, to the electrode patch, thus coupling the tubes to surface 70 of plate 54. (Adhesive patch-sheet 60 may interpose between the tubes and surface 70.)

Next, at a connecting step 90, the fluid conduits are connected to the tube-ends of tubes 62, and the electrical connections to the electrode patch are established. Subsequently, at a third removing step 94, second outer adhesive-sheet backing 74o2 is removed from the second outer adhesive portion of adhesive sheet 72. The patch and the second outer adhesive portion are then adhered to skin of the subject, at a third adhering step 96. For example, a backing may be removed from adhesive patch-sheet 60, and then adhesive patch-sheet 60, along with the second outer adhesive portion of adhesive sheet 72, may be adhered to the subject's skin. (In some embodiments, at least some parts of connecting step 90 are performed only after the patch is already stuck to the subject.)

Following the adhering of the patch to the subject, the passing of fluid through the tubes is begun, at a fluid-passing-beginning step 98. In particular, fluid is passed from one of the fluid conduits, through the tubes, to the other fluid conduit, such that the fluid passes through the electrode patch. Finally, the ablation procedure is begun, at an ablation-beginning step 100. Subsequently, during the procedure, while electric current passes through the electrically-conducting plate belonging to the patch, the passage of fluid through the tubes is continued, such that the fluid evacuates heat from the electrically-conducting plate. For example, as described above with reference to FIGS. 2B-C, the fluid may be continually cycled through the tubes.

For integrated patch 30a, method 78 may be simplified, given that fewer steps are required to prepare the patch for the procedure. In particular, the method may begin with connecting step 90. Following connecting step 90, the patch may be adhered to the subject, and fluid-passing-beginning step 98 and ablation-beginning step 100 may then be performed.

It will be appreciated by persons skilled in the art that the present invention is not limited to what has been particularly shown and described hereinabove. Rather, the scope of embodiments of the present invention includes both combinations and subcombinations of the various features described hereinabove, as well as variations and modifications thereof that are not in the prior art, which would occur to persons skilled in the art upon reading the foregoing description. Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

1. Apparatus, comprising:
at least one electrically-conducting plate, configured for placement on a body of a subject; and
one or more tubes coupled to a surface of the electrically-conducting plate, the tubes being configured to carry a fluid over the electrically-conducting plate while the electrically-conducting plate is on the body of the subject and electric current passes through the electrically-conducting plate.

2. The apparatus according to claim 1, wherein the tubes comprise a first tube-end and a second tube-end that protrude from the electrically-conducting plate, and wherein the tubes are configured to carry the fluid, over the plate, from the first tube-end to the second tube-end.

3. The apparatus according to claim 2, wherein the first tube-end and the second tube-end comprise respective threaded connectors.

4. The apparatus according to claim 1, wherein the tubes are interconnected, such as to define a tube network.

5. The apparatus according to claim 4, wherein the tubes comprise:
a first tube coupled to a first edge of the surface;
a second tube coupled to a second edge of the surface that is opposite the first edge; and
a plurality of third tubes that connect the first tube to the second tube, such that the fluid flows between the first tube and the second tube via the third tubes.

6. The apparatus according to claim 1, further comprising one or more temperature sensors coupled to the electrically-conducting plate.

7. Apparatus for use with a patch that includes an electrically-conducting plate, the apparatus comprising:
one or more tubes, configured to carry a fluid over the electrically-conducting plate while the patch is coupled to a body of a subject, and while electric current passes through the electrically-conducting plate;
an adhesive sheet, comprising:
an inner adhesive portion, configured to couple to the tubes; and
an outer adhesive portion, which at least partly surrounds the inner adhesive portion and is configured to couple the tubes to a surface of the electrically-conducting plate by adhering to the patch while the tubes are coupled to the inner adhesive portion;
an inner adhesive-sheet backing, which covers the inner adhesive portion; and
an outer adhesive-sheet backing, which covers the outer adhesive portion.

8. The apparatus according to claim 7, further comprising a tube-mounting sheet, wherein the tubes are mounted on the tube-mounting sheet, and wherein the inner adhesive portion is configured to couple to the tubes by adhering to the tube-mounting sheet.

9. The apparatus according to claim 8, further comprising one or more temperature sensors coupled to the tube-mounting sheet.

10. The apparatus according to claim 7,
wherein the outer adhesive portion is a first outer adhesive portion and the outer adhesive-sheet backing is a first outer adhesive-sheet backing,
wherein the adhesive sheet further comprises a second outer adhesive portion, which at least partly surrounds the first outer adhesive portion and is configured to adhere to skin of the subject, and
wherein the apparatus further comprises a second outer adhesive-sheet backing, which covers the second outer adhesive portion.

11. The apparatus according to claim 7, wherein the tubes are interconnected, such as to define a tube network.

12. The apparatus according to claim 11, wherein the tubes comprise:
a first tube;
a second tube that is opposite the first tube; and
a plurality of third tubes that connect the first tube to the second tube.

13. A method, comprising:
connecting a first fluid conduit to a first tube-end of one or more tubes coupled to a surface of an electrically-conducting plate;
connecting a second fluid conduit to a second tube-end of the tubes; and
while the electrically-conducting plate is on a body of a subject and electric current passes through the electrically-conducting plate, passing a fluid from the first fluid conduit, through the tubes, to the second fluid conduit, such that the fluid evacuates heat from the electrically-conducting plate.

14. The method according to claim 13, wherein passing the fluid through the tubes comprises passing the fluid through the tubes by, using a pump, pumping the fluid from a fluid source through the first fluid conduit, such that the fluid flows through the first fluid conduit, through the tubes, and through the second fluid conduit to a drain.

15. The method according to claim 13, wherein passing the fluid through the tubes comprises passing the fluid through the tubes by cyclically pumping the fluid through the tubes.

16. The method according to claim 15, wherein cyclically pumping the fluid through the tubes comprises cyclically pumping the fluid through the tubes by cyclically pumping the fluid from a fluid bag through the first fluid conduit, such that the fluid flows through the first fluid conduit, through the tubes, and through the second fluid conduit to the fluid bag.

17. The method according to claim 15, further comprising cooling the fluid while the fluid flows through the second fluid conduit.

18. The method according to claim 13,
wherein the tubes include:
a first tube that runs along a first edge of the surface and terminates at the first tube-end,
a second tube that runs along a second edge of the surface, which is opposite the first edge, and terminates at the second tube-end, and
a plurality of third tubes that connect the first tube to the second tube, and
wherein passing the fluid through the tubes comprising passing the fluid between the first tube and the second tube via the third tubes.

19. The method according to claim 13, wherein the plate is included in a patch, and wherein the method further comprises, prior to passing the fluid through the tubes, adhering the patch to skin of the subject.

20. The method according to claim 19, further comprising coupling the tubes to the surface of the electrically-conducting plate, by adhering an adhesive sheet, over the tubes, to the patch.

21. The method according to claim 20, further comprising, prior to coupling the tubes to the surface of the electrically-conducting plate, coupling the adhesive sheet to the tubes.

22. The method according to claim 21, wherein the tubes are mounted on a tube-mounting sheet, and wherein coupling the adhesive sheet to the tubes comprises coupling the adhesive sheet to the tubes by adhering the adhesive sheet to the tube-mounting sheet.

23. The method according to claim 20, further comprising adhering the adhesive sheet to skin of the subject.

24. The method according to claim 13, wherein passing the fluid through the tubes comprises controlling a rate at which the fluid is passed through the tubes responsively to a sensed temperature of the electrically-conducting plate.
